(19) United States
(12) Patent Application Publication
(10) Pub. No.: US 2017/0327504 A1
(43) Pub. Date: Nov. 16, 2017

(54) Synthesis of Substituted 1H-Pyrazolo[3,4-D]Pyrimidines

(71) Applicant: Sandoz AG, Basel (CH)

(72) Inventors: Christopher Rose, Kundl (AT); Herbert Silberger, Kundl (AT); Erwin Schreiner, Kundl (AT); Wolfgang Felzmann, Kundl (AT); Nenad Maras, Kundl (AT)

(73) Assignee: Sandoz AG, Basel (CH)

(21) Appl. No.: 15/523,067

(22) PCT Filed: Oct. 28, 2015
(86) PCT No.: PCT/EP2015/074956

§ 371 Date: Apr. 28, 2017

(30) Foreign Application Priority Data
Oct. 30, 2014 (EP) 14191001.8

(51) Int. Cl. C07D 487/04

(52) U.S. Cl. CPC C07D 487/04

(57) ABSTRACT

The present invention refers to the synthesis and intermediates of substituted bicyclic compounds by using a central 1H-pyrazolo[3,4-d]pyrimidine of formula (I), which is assembled starting from 4,6-dichloropyrimidine carboxylic acid. The invention in particular refers to the synthesis of the Bruton's tyrosine kinase (Btk) inhibitor 1-((R)-3-(4-amino-3-(4-phenoxyphenyl)-1H-pyrazolo[3,4-d]pyrimidin-1-yl)piperidin-1-yl)prop-2-en-1-one(ibrutinib) and its synthesis intermediates.

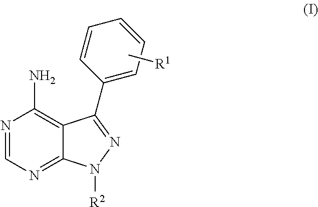

### FIELD OF THE INVENTION

The present invention refers to the synthesis and intermediates of substituted bicyclic compounds by using a central 1H-pyrazolo[3,4-d]pyrimidine, which is assembled starting from 2,6-dichloropyrimidine carboxylic acid. The invention in particular refers to the synthesis of the Bruton's tyrosine kinase (Btk) inhibitor 1-((R)-3-(4-amino-3-(4-phenoxyphenyl)-1H-pyrazolo[3,4-d]pyrimidin-1-yl)piperidin-1-yl)prop-2-en-1-one (ibrutinib) and its synthesis intermediates.

### BACKGROUND OF THE INVENTION

Inhibitors of kinases involved in mediating or maintaining disease states represent novel therapies for various disorders, such as hyperproliferative diseases and cancer. Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

1-((R)-3-(4-amino-3-(4-phenoxyphenyl)-1H-pyrazolo[3,4-d]pyrimidin-1-yl)piperidin-1-yl)prop-2-en-1-one is also known by its IUPAC name as 1-{(3)-3-[4-amino-3-(4-phenoxyphenyl)-1H-pyrazolo[3,4-d]pyrimidin-1-yl]piperidin-1-yl}prop-2-en-1-one or 2-propen-1-one-1-[(3R)-3-[4-amino-3-(4-phenoxyphenyl)-1H-pyrazolo[3,4-d]pyrimidin-1-yl]-1-piperidinyl], and has been given the USAN name “Ibrutinib”, which will be used further in the document and refers to the compound with the following structure:

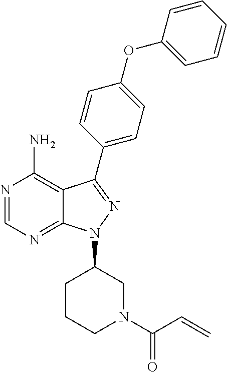 [SMILES: C=CC(=O)N1CCC[C@@H](n2nc(-c3ccc(Oc4ccccc4)cc3)c3c(N)ncnc32)C1]

Ibrutinib is an orally-administered, selective and covalent irreversible inhibitor of the enzyme Bruton's tyrosine kinase. It was first disclosed in WO 2008/039218, and has been shown to be highly clinically efficacious in relapsed/refractory chronic lymphocytic leukemia (CLL) and mantle cell lymphoma (see e.g. Burger et al., Leukemia & Lymphoma (2013), 54(11), 2385-91).

Ibrutinib has been reported to promote apoptosis, inhibit proliferation, and also prevent CLL cells from responding to survival stimuli provided by the microenvironment. Treatment of activated CLL cells with ibrutinib resulted in inhibition of Btk tyrosine phosphorylation and also effectively abrogated downstream survival pathways activated by this kinase. Additionally, ibrutinib inhibited proliferation of CLL cells in vitro, effectively blocking survival signals provided externally to CLL cells from the microenvironment. Further, ibrutinib has been reported to inhibit cellular adhesion following stimulation at the B cell receptor. Together, these data are consistent with a mechanistic model whereby ibrutinib blocks B cell receptor signaling, which drives cells into apoptosis and/or disrupts cell migration and adherence to protective tumour microenvironments.

WO 01/019829 describes a general synthesis for substituted 1H-pyrazolo[3,4-d]pyrimidines. A Knoevenagel-condensation of phenoxybenzoic acid chloride and malonic acid dinitrile furnishes the enole, which is subsequently methylated using hazardous trimethylsilyldiazomethane. The pyrazole- and pyrimidine ring systems are then assembled via two successive condensation reactions.

WO 2008/039218 and WO 2008/121742 describe a synthesis of ibrutinib, with the 1H-pyrazolo[3,4-d]pyrimidine being assembled according to WO 0119829 A2. The coupling of the chiral piperidine building block is accomplished via a Mitsunobu reaction, generating a large waste stream. Ibrutinib is then obtained after a final protecting group manipulation (Boc-removal followed by coupling with acryloyl chloride). In total, the described process comprises an uneconomical high number of eight process steps.

In CN 103121999 a 1H-pyrazolo[3,4-d]pyrimidine is obtained via palladium-catalyzed cross-coupling of a 3-Halo-1H-pyrazolo[3,4-d]pyrimidine with phenoxyphenyl boronic acid—both of which being very expensive chemicals. In contrast to WO08039218, an additional trifluoroacetyl is introduced which has to be removed at the end of the synthetic sequence.

CN 103626774 discloses a synthesis starting with a Knoevenagel-condensation of phenoxybenzoic acid chloride and malonic acid dinitrile, furnishing an enol-ether after methylation with dimethyl sulphate. The pyrazole-ring system is assembled via condensation with a piperidinyl hydrazine. A final condensation reaction then gives rise to Ibrutinib. WO2014/139970 describes a similar sequence, with emphasis on the synthesis of the complex piperidinyl hydrazine derivatives used for the pyrazole synthesis.

However, the preparation of the chiral piperidinyl hydrazine derivative requires a costly chiral chromatography step. Furthermore, the final step has the same drawbacks as described in WO 2008/039218.

In view of the above described prior art, a need exists for a more efficient synthetic route for the synthesis of substituted 1H-pyrazolo[3,4-d]pyrimidines, such as ibrutinib and derivatives thereof. In particular, the synthesis should be more economical than the synthetic routes of the prior art, i.e. should need only a reduced number of process steps, starting from cheap materials. Further, a synthesis free from use or generation of hazardous materials is desired. In particular, it should avoid the generation of large waste streams, for example by avoiding an uneconomical Mitsunobu reaction. It is therefore desired to find a new synthesis for ibrutinib and its derivatives, which overcomes the disadvantages of the prior art processes.

Further, a need exists in the art for the synthesis of novel substituted 1H-pyrazolo[3,4-d]pyrimidines to find novel therapeutic agents active as receptor or non-receptor tyrosine kinase inhibitors, in particular, Btk inhibitors.

It is has surprisingly been found in the present invention that the problems of the prior art can be solved by the provision of a synthesis for substituted 1H-pyrazolo[3,4-d]pyrimidines, such as ibrutinib and derivatives thereof, using a central 1H-pyrazolo[3,4-d]pyrimidine, which is assembled starting from 2,6-dichloropyrimidine carboxylic acid, as described herein.

### DESCRIPTION OF THE INVENTION

### Definition of Terms

An “alkyl” group refers to a hydrocarbon group which is not aromatic. The alkyl moiety may be a “saturated alkyl” group, which means that it does not contain any carbon-carbon double or triple bonds. The alkyl moiety may also be an “unsaturated alkyl” moiety, which means that it contains at least one carbon-carbon double or triple bond. “Unsaturated alkyl” moieties containing at least one carbon-carbon double bond are referred to as an “alkene” moiety. “Unsaturated alkyl” moieties containing at least one carbon-carbon triple bond are referred to as an “alkyne” moiety. The alkyl moiety, whether saturated or unsaturated, may be branched or straight chain.

The (saturated) “alkyl” group may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, isopropyl, n-butyl, isobutyl, sec-butyl, and t-butyl. Typical alkyl groups include, but are not limited to, methyl, ethyl, propyl, isopropyl, butyl, isobutyl, tertiary butyl, pentyl, 2-methylbutyl, 3-methylbutyl, 3,3-dimethylpropyl, hexyl, 2-methylpentyl, 3,3-dimethylbutyl, 2,3-dimethyl butyl and the like. Alkyl groups can be substituted or unsubstituted.

As indicated above the term “alkenyl” refers to a type of unsaturated alkyl group that is not part of an aromatic group. Alkenyl groups may have 2 to 10 carbons. The alkenyl moiety may be branched or straight chain. Alkenyl groups can be optionally substituted. Non-limiting examples of an alkenyl group include —C(CH₃)═CH₂, —CH═CH₂, —CH═C(CH₂CH₃)₂, —CH═CHCH₃, —C(CH₃)═CHCH₃. As indicated above the term “alkynyl” refers to a type of unsaturated alkyl group in which two atoms of the alkyl group form a triple bond. Alkynyl groups may have 2 to 10 carbons. The alkynyl moiety may be branched or straight chain. Alkynyl groups can be optionally substituted. Non-limiting examples of an alkynyl group include, but are not limited to, —C≡CH, —C≡CCH₃, —C≡CCH₂CH₃.

A heteroalkyl group refers to an alkyl group as defined above wherein at least one carbon atom is substituted with a heteroatom such as nitrogen, oxygen, sulphur and/or phosphorus.

A “cycloalkyl” group refers to a hydrocarbon group, which is not aromatic and wherein at least three carbon atoms are forming a ring. As used herein, the term “ring” refers to any covalently closed structure. Rings can be monocyclic or polycyclic. The cycloalkyl moiety may be a “saturated cycloalkyl” group, which means that it does not contain any carbon-carbon double or triple bonds. The cycloalkyl moiety may also be an “unsaturated cycloalkyl” moiety, which means that it contains at least one carbon-carbon double or triple bond. The (saturated) “cycloalkyl” moiety may have 3 to 12 carbon atoms. The cycloalkyl group of the compounds described herein may be designated as “C₃-C₁₂ cycloalkyl” or similar designations. By way of example only, “C₃-C₅ cycloalkyl” indicates that there are three to five carbon atoms in the cycloalkyl ring, i.e. the cycloalkyl ring is selected from among cyclopropyl, cyclobutyl, and cyclopentyl. Typical cycloalkyl groups include, but are in no way limited to cyclopropyl, cyclobutyl, and cyclopentyl, cyclohexyl, cycloheptyl, cyclooctyl and the like. Cycloalkyl groups can be substituted or unsubstituted.

As indicated above a “cycloalkenyl” group refers to an unsaturated cycloalkyl group, wherein at least five carbon atoms are forming a ring. The “cycloalkenyl” moiety may have 5 to 12 carbon atoms. The cycloalkenyl group of the compounds described herein may be designated as “C₅-C₁₂ cycloalkenyl” or similar designations. By way of example only, “C₅-C₈ cycloalkenyl” indicates that there are five to eight carbon atoms. Cycloalkenyl groups can be substituted or unsubstituted. Typical cycloalkenyl groups include, but are in no way limited to cyclopentenyl, cyclohexenyl, cycloheptenyl, cyclooctenyl and the like.

A heterocycloalkyl group refers to a cycloalkyl group as defined above wherein at least one carbon atom being part of the ring is a heteroatom such as nitrogen, oxygen sulphur and/or phosphorus.

The term “aryl” group refers to a residue with an aromatic skeletal structure, wherein the ring atoms of the aromatic skeletal structure are carbon atoms. The term “aromatic” refers to a planar ring having a delocalized [pi]-electron system containing 4n+2 [pi] electrons, where n is an integer. The aryl group can be formed from five, six, seven, eight, nine, or more than nine atoms. Aryl groups can be optionally substituted. The aryl groups can be monocyclic or polycyclic (i.e., rings which share adjacent pairs of carbon atoms) groups.

Examples of aryl groups include, but are not limited to phenyl, biphenyl, naphthyl, binaphthyl, pyrenyl, azulenyl, phenanthryl, anthracenyl, fluorenyl, and indenyl.

The term heteroaryl group refers to an aryl group as defined above wherein at least one carbon atom being part of the aromatic skeletal ring structure is a heteroatom such as nitrogen, oxygen, sulphur and/or phosphorus.

Examples of heteroaryl groups include, but are not limited to pyrrolyl, imidazolyl, furyl, thienyl, oxazolyl, thiazolyl, thiadiazolyl, tetrazolyl, pyridyl, pyrimidyl, triazolyl, indolyl, isoindolyl, benzofuranyl, dibenzofuranyl, benzothienyl, benzimidazolyl.

The above (hetero)alkyl, (hetero)cycloalkyl and (hetero)aryl groups can optionally be substituted with one or more substituents. Examples of substituents are alkyl, heteroalkyl, cycloalkyl, heterocycloalkyl, aryl, heteroaryl, alkoxy, cycloalkoxy, aryloxy, alkylthio, cycloalkylthio, arylthio, alkylsulfoxide, arylsulfoxide, alkylsulfone and arylsulfone.

Further Examples of substituents are cyano, nitro, halogen, hydroxy or protected hydroxy groups, amines or protected amines, monoalkyl amines or protected monoalkyl amines, monoarylamines or protected monoarylamines, dialkylamines, diarylamines, amides and esters.

An “amide” is a chemical moiety is with the functional group —C(O)NR₂, where R refers to H or organic groups, and preferably refers to a chemical moiety with the formula —C(O)NHR or —NHC(O)R^{A}, where R^{A} may be selected from among (hetereo)alkyl, (hetero)aryl and (hetero)cycloalkyl as described herein.

The term “ester” refers to a chemical moiety with formula —COOR^{E}, where R^{E} is selected from among (hetereo)alkyl, (hetero)cycloalkyl and (hetero)aryl groups as described herein.

The term “halogen” comprises chloro, bromo and iodo.

The term “monoalkylamine” refers to the —NH(alkyl), where the alkyl groups are as defined herein.

The term “dialkylamine” refers to the —N(alkyl)₂, where the alkyl groups are as defined herein or further when taken together with the N atom to which they are attached, can optionally form a cyclic ring system.

The term “diarylamine” refers to the —N(aryl)₂, where the aryl groups are as defined herein.

Protection groups for amines or mono-substituted amines are for example Boc (tertbutyloxycarbonyl), Z or Cbz (benzyloxycarbonyl), benzyl, benzhydryl and Fmoc (fluorenylmethylenoxycarbonyl).

Protection groups for hydroxyl groups are for example esters, such as benzoic acid esters or pivalic acid esters, and trisubstituted silylethers, such as trimethylsilylether, triethylsilylether, tert-butyldimethylsilylether and tert-butyl diphenylsilylether.

Further examples of suitable amine or hydroxyl protecting groups can be found in Greene, P. G. M.; Wuts, T. W. Greene's Protective Groups in Organic Synthesis, 4^{th} Edition, 2007, John Wiley & Sons, Hoboken, N.J.

### DETAILED DESCRIPTION OF EMBODIMENTS OF THE INVENTION

In a first embodiment, the present invention refers to a process for the preparation of a compound of formula (I),

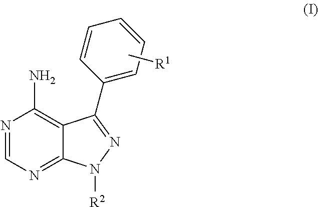

comprising reacting a compound of formula (II)

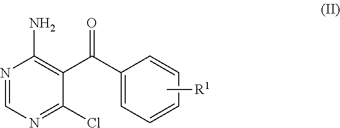

with a compound of formula (III)

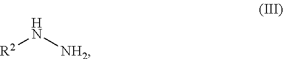

optionally in the presence of an alkaline substance, wherein
R¹ is selected from OR⁴, SR⁴, NR⁴R⁵ and halogen, preferably is OR⁴, and most preferably is OPh.
R² is selected from hydrogen, substituted or unsubstituted alkyl, substituted or unsubstituted cycloalkyl and substituted or unsubstituted heterocycloalkyl, preferably substituted or unsubstituted cycloalkyl and substituted or unsubstituted heterocycloalkyl, and
R⁴ and R⁵ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl, preferably is substituted or non-substituted aryl.

In a preferred embodiment, R¹ is OR⁴ and R⁴ is a substituted or non-substituted aryl.

R¹ may be in ortho, meta or para position, but is preferably in ortho or para position, most preferably in para position. In case of multiple substituents R¹, it is preferred that at least one R¹ is in para position.

In a preferred aspect of the invention, the compound according to formula (III) is represented by the following formula (IIIa)

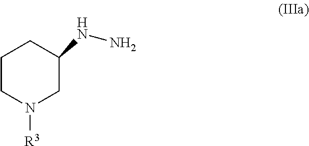

wherein R³ is selected from hydrogen, a group selected from carbamoyl, substituted or non-substituted benzyl and substituted or non-substituted silyl, and C(O)—R⁶, wherein
R⁶ is selected from hydrogen, substituted or non-substituted alkyl, substituted or non-substituted alkenyl, substituted or non-substituted heteroalkyl, substituted or non-substituted heteroalkenyl, substituted or non-substituted cycloalkyl, substituted or non-substituted heterocycloalkyl, substituted or non-substituted cycloalkenyl, substituted or non-substituted heterocycloalkenyl, substituted or non-substituted aryl, and substituted or non-substituted heteroaryl.

In a further preferred aspect, the compound according to formula (III) is represented by the following formula (IIIb)

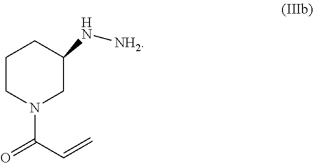

The alkaline substance is typically selected from amino-group containing substances such as triethylamine, ethyl-diisopropylamine or pyridine, and preferably is triethylamine.

In a typical example, the reaction is carried out using 2.5 to 4.5 eq. of compound III relative to compound II. Suitable solvents for this process, without being limited to, include methyl-tetrahydrofuran, methanol, ethanol, 2-propanol, 1-butanol, diethylcarbonate, acetonitrile or dimethylsulfoxide, optionally in the presence of an alkaline substance as described herein, preferably triethylamine. The reaction is typically carried out at elevated temperature, preferably 50° C. to 120° C., even more preferably 60° C. to 100° C., further preferably 70° C. to 90° C. After completed reaction, the product may be isolated by column chromatography.

In a further preferred aspect, the compound of formula (II) is obtained by monoamination of a compound of formula (IV) as described in the following.

Thus, in another embodiment, the present invention relates to process for the preparation of a compound according to formula (II), comprising

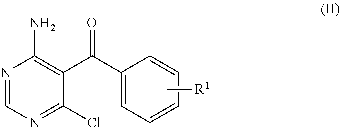

monoamination of a compound of formula (IV)

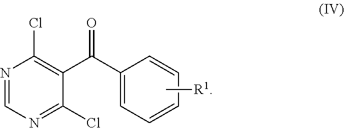

Monoamination is typically carried out in the presence of ammonia and with a temperature in a range of 20 to 100° C., preferably 50 to 90° C., most preferably 60 to 80° C.

In a typical example, the reaction is carried out using 5 to 10 eq. of ammonia relative to compound IV. Suitable sources of ammonia include methanolic ammonia solution, aqueous ammonia solution or gaseous ammonia. Suitable solvents for this process, without being limited to, include tetrahydrofuran, methanol and toluene. The reaction is typically carried out with a temperature in a range of 20 to 100° C., preferably 50 to 90° C., most preferably 60 to 80° C. After completed reaction, the product may be isolated by evaporation of the solvent optionally followed by crystallization.

In a further preferred aspect, the compound of formula (IV) is obtained by reacting a compound of formula (V) with a compound of formula (VI) in the presence of a Lewis acid as described in the following.

Thus, in another embodiment, the present invention relates to process for the preparation of a compound according to formula (IV) by reacting a compound according formula (V)

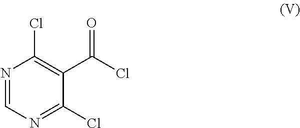 [SMILES: O=C(Cl)c1c(Cl)ncnc1Cl]

with a compound according to formula (VI)

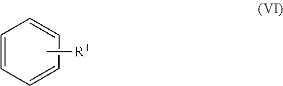

in the presence of a Lewis acid.

Generally, a Lewis acid can be regarded as a molecular entity being an electron pair acceptor and able to react with a Lewis base being an electron pair donor. By reacting a Lewis acid with a Lewis base a Lewis adduct is formed by sharing the electron pair provided by the Lewis base.

In a preferred embodiment the Lewis acid is selected from AlX₃, TiX₄, ZrX₄, HfX₄, SnX₄, FeX₃, BX₃, CuX₂, VX₄, ScX₃, YX₃, LnX₃, preferably selected from AlX₃, TiX₄, ZrX₄, SnX₄, ScX₃, BX₃, wherein X is halogen, a substituted or unsubstituted alkylsulfonyl group, a substituted or unsubstituted arylsulfonyl group, a substituted or unsubstituted alkoxy group or a substituted or unsubstituted aryloxy group. Preferably, the Lewis acid is AlCl₃ or FeCl₃, most preferably is AiCl₃.

In a typical example, the reaction is carried out using 2.6 eq. of compound VI relative to compound V in the presence of 2.5 eq. of a Lewis-acid. Suitable Lewis-acids for this reaction include AlCl₃ and FeCl₃. Most preferred is AlCl₃. Suitable solvents for this process include dichloromethane and nitrobenzene. Most preferred is dichloromethane. The reaction is preferably carried out at a temperature of 50° C. After completed reaction, the product may be isolated by chromatographic purification or crystallization.

In a further preferred aspect, the compound of formula (V) is obtained by reacting a compound of formula (VII) with a chlorinating agent as described in the following.

Thus, in another embodiment, the present invention relates to process for the preparation of a compound according to formula (V) by reacting a compound according formula (VII)

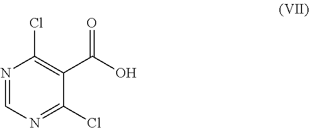 [SMILES: O=C(O)c1c(Cl)ncnc1Cl]

with a chlorinating agent.

Suitable chlorinating agents are selected from one or more of (COCl)₂/DMF, SOCl₂, PCl₅, PCl₃, POCl₃/DMF, 1-Chloro-N,N,2-trimethyl-1-propenylamine. Preferably, the chlorinating agent is (COCl)₂/DMF.

In a typical example, the reaction is carried out using 1 to 1.2 eq. of oxalyl chloride relative to compound VII and in the presence of 3 to 5 mol % of dimethylformamide. Suitable solvents for this process, without being limited to, include tetrahydrofuran, ethylacetate, diethylether, dimethylcarbonate and dichloromethane. The reaction is typically carried out with a temperature in a range of 20 to 25° C., preferably at 25° C. After completed reaction, the product may be isolated by evaporation of the solvent.

In a particularly preferred aspect of the invention, R¹ is OPh, and compound (III) is represented by the compound of formula (IIIa). Thus, the process of this aspect relates to the preparation of ibrutinib, which is represented by the following formula (Ia)

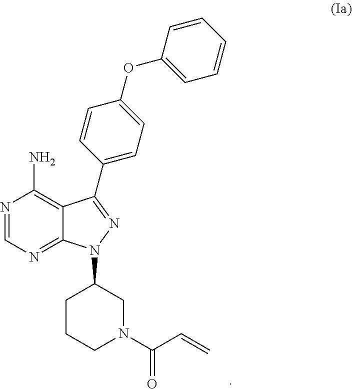 [SMILES: C=CC(=O)N1CCC[C@@H](n2nc(-c3ccc(Oc4ccccc4)cc3)c3c(N)ncnc32)C1]

In another embodiment, the present invention relates to a compound represented by the formula (IIa)

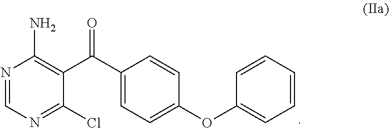 [SMILES: Nc1ncnc(Cl)c1C(=O)c1ccc(Oc2ccccc2)cc1]

In another embodiment, the present invention relates to a compound represented by the formula (IVa)

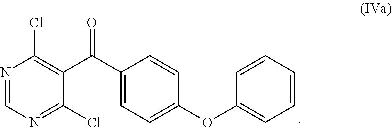 [SMILES: O=C(c1ccc(Oc2ccccc2)cc1)c1c(Cl)ncnc1Cl]

In another embodiment, the present invention relates to the use of compounds (IIa) and/or (IVa) for the preparation of ibrutinib.

Thus, in a further preferred embodiment, the present invention relates to the preparation of a compound of formula (I),

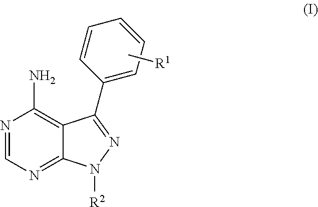

comprising the steps of:
a) reacting a compound according to formula (VII) with a chlorinating agent to obtain a compound according to formula (V),
b) reacting a compound according to formula (V) with a compound according to formula (VI) in the presence of a Lewis acid to obtain a compound according to formula (IV),
c) monoamination of a compound according to formula (IV) to obtain a compound according to formula (II), and
d) reacting a compound according to formula (II) with a compound of formula (III) to obtain a compound according to formula (I), optionally in the presence of an alkaline substance,

wherein R¹, R², R³, R⁴ and R⁵ of the formulae (I) to (VII) are as defined above.

In another embodiment, the present invention relates to a process for the synthesis of compound (VIII)

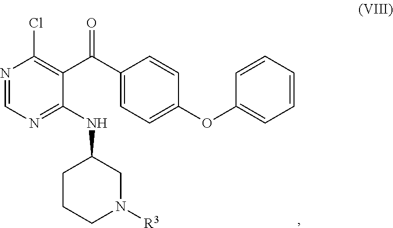

comprising treating a compound of formula (IVa)

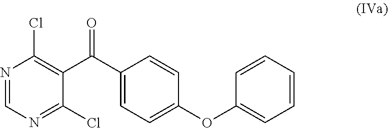 [SMILES: O=C(c1ccc(Oc2ccccc2)cc1)c1c(Cl)ncnc1Cl]

with a primary amine of the formula (IX):

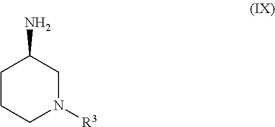

optionally in the presence of an alkaline substance.
wherein R³ is selected from carbamoyl, substituted or non-substituted benzyl and substituted or non-substituted silyl, and C(O)—R⁶, wherein
R⁶ is selected from hydrogen, substituted or non-substituted alkyl, substituted or non-substituted heteroalkyl, substituted or non-substituted cycloalkyl, substituted or non-substituted cycloalkenyl, substituted or non-substituted heterocycloalkyl, substituted or non-substituted heterocycloalkenyl, substituted or non-substituted aryl, substituted or non-substituted heteroaryl, and in particular may be a group selected from

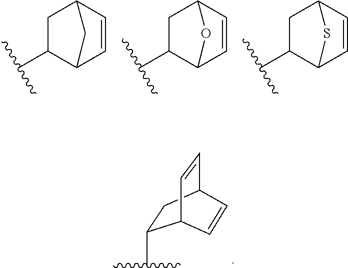

In a typical example, the reaction is carried out using 1.2 to 5 eq. of compound IX relative to compound IVa, optionally in the presence of an alkaline substance. Suitable solvents for this process, without being limited to, include 2-methyltetrahydrofuran, tetrahydrofuran and toluene. The reaction is typically carried out with a temperature in a range of 70 to 110° C., preferably in a range of 80° C. to 100° C., further preferably at 90° C. After completed reaction, the product may be isolated by chromatographic purification.

In another embodiment, the present invention relates to a compound represented by the following formula (VIII), wherein R³ is as defined above.

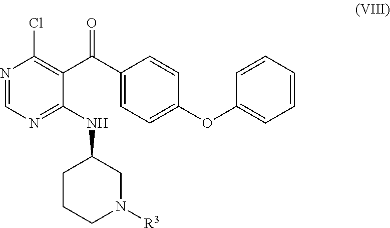

In another embodiment, the present invention relates to a compound represented by one of the following formulae (Ib), (Ic) or (Id).

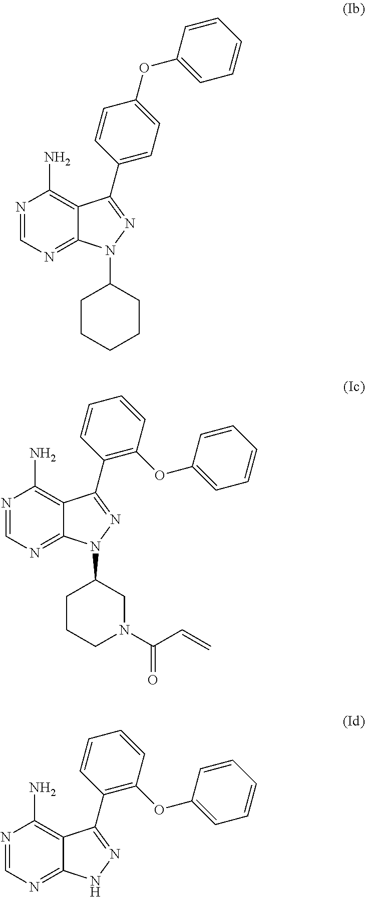 [SMILES: Nc1ncnc2c1c(-c1ccc(Oc3ccccc3)cc1)nn2C1CCCCC1 ; C=CC(=O)N1CCC[C@@H](n2nc(-c3ccccc3Oc3ccccc3)c3c(N)ncnc32)C1 ; Nc1ncnc2[nH]nc(-c3ccccc3Oc3ccccc3)c12]

In another embodiment, the present invention relates to a compound represented by one of the following formulae (IVb) or (IIb).

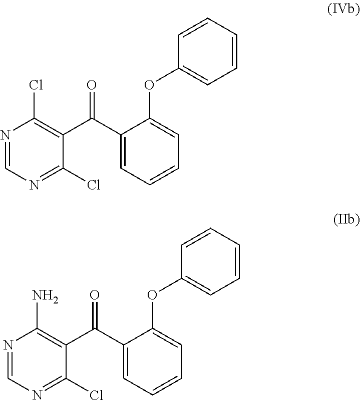 [SMILES: O=C(c1ccccc1Oc1ccccc1)c1c(Cl)ncnc1Cl ; Nc1ncnc(Cl)c1C(=O)c1ccccc1Oc1ccccc1]

Compounds of the above formulae Ic, Id, IVb and IIb may be obtained as byproducts during the herein disclosed synthesis of Ibrutinib or derivatives thereof. They are typically obtained in an amount of less than 15 wt. %, preferably less than 10 wt. %, more preferably less than 5 wt. %, on the basis of the total amounts of reaction products obtained in the respective process as described herein.

In another embodiment, the present invention relates to a compound represented by one of the following formulae (X), (XI) and (XII), wherein R³ is as defined above.

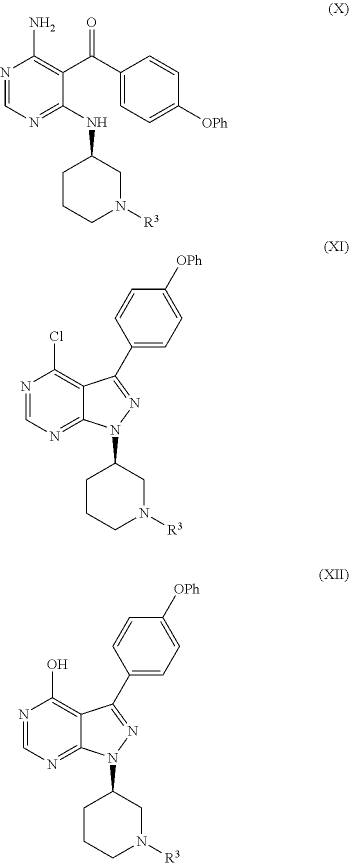

In a typical example, the reaction for producing a compound of the above formula (X) is carried out using 4.5 eq. of compound IX relative to compound IIa, optionally in the presence of an alkaline substance. Suitable solvents for this process, without being limited to, include 2-methyltetrahydrofuran and tetrahydrofuran. The reaction is typically carried out with a temperature in a range of 70 to 90° C., preferably at 90° C. After completed reaction, the product may be isolated by chromatographic purification.

The synthetic route of the present invention for the synthesis of substituted 1H-pyrazolo[3,4-d]pyrimidines, and in particular for the synthesis of ibrutinib and derivatives thereof comprises fewer synthetic steps as the prior art processes, thus is more convergent and more efficient, and in particular avoids an uneconomical Mitsunobu reaction. Moreover, no hazardous reagents such as trimethylsilyldiazomethane are required. Moreover, it starts from cheap materials. Further, it employs less protecting group manipulations, is free of phosphines, or transition metal mediated couplings, which may contaminate the active ingredient. Moreover, it is more economical than the prior art syntheses, as it eliminates the generation of hazardous materials and large waste streams, for example, no toxic acrylate reagents are used in the final synthesis step, thereby leading to the efficient synthesis of ibrutinib and derivatives thereof. Further particular, the process of the present invention can efficiently deplete quaternary ammonium salts used as phase-transfer catalysts, which may otherwise be present in the final product as impurity. Further, the final active acrylamide compound can be liberated under neutral conditions avoiding any basic or acidic conditions which can lead to the formation of degradation- or by-products.

Further, the synthesis as described herein allows modular access to substituted N-alkyl pyrazolo pyrimidines, in turn enabling library synthesis for new drug identification.

### Dosage Form

Ibrutinib or any of the substituted 1H-pyrazolo[3,4-d]pyrimidines prepared by the above-described processes may be used for the manufacture of a pharmaceutical composition. In a further embodiment, the present invention relates to a compound as described herein for use as a medicament. In particular, the present invention relates to a compound as described herein for use in the treatment of cancer.

In a further embodiment, the present invention relates to a pharmaceutical composition comprising a compound prepared by the processes as described herein, and in particular relates to a pharmaceutical composition comprising ibrutinib or one of its derivatives as prepared by a process as described herein.

The pharmaceutical composition typically comprises 1.0 to 1000 mg, preferably comprises 10 to 800 mg, most preferably comprises 50 to 550 mg of the compounds prepared by the above-described processes, such as ibrutinib, particularly amorphous ibrutinib.

The pharmaceutical composition may further comprise one or more pharmaceutically acceptable additives, such as binders, carriers, stabilizers, diluents, dispersing agents, suspending agents, thickening agents, and/or excipients. Suitable excipients include, for example, fillers such as sugars, including lactose, sucrose, mannitol, or sorbitol; cellulose preparations such as, for example, maize starch, wheat starch, rice starch, potato starch, gelatine, gum tragacanth, methylcellulose, micro crystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

The pharmaceutical composition facilitates administration of the compound to a mammal, preferably to a human. Ibrutinib or anyone of the compounds as described herein can be used singly or in combination with one or more therapeutic agents as components of mixtures.

The pharmaceutical composition is typically a solid oral dosage form. It may be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations. Preferably, the pharmaceutical dosage form is a tablet or capsule.

The pharmaceutical compositions may be manufactured in a conventional manner, such as, by way of example only, by means of conventional mixing, dissolving, granulating, dragee-making, levigating, emulsifying, encapsulating, entrapping or compression processes.

In another embodiment, the compounds prepared by the above-described processes, such as ibrutinib or a derivative thereof, may be used in the treatment of cancer. In particular, cancer may be a B cell malignancy, preferably selected from chronic lymphocytic leukemia (CLL)/small lymphocytic lymphoma (SLL), mantle cell lymphoma (MCL), indolent non-Hodgkins's lymphoma, diffuse large B Cell lymphoma (DLBCL), multiple myeloma (MM), marginal zone lymphoma (NHL), hairy cell leukemia, acute lymphocyte leukemia (ALL), and breast cancer.

### Examples

In the following, the present invention will further be described by the way of non-limiting examples.

### Example 1: Synthesis of 4,6-dichloropyrimidine-5-carboxilic acid chloride

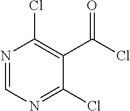 [SMILES: O=C(Cl)c1c(Cl)ncnc1Cl]

In a three-necked round-bottom flask with nitrogen-inlet, 4,6-dichloropyrimidine-5-carboxylic acid (3.80 g, 1 eq.) was dissolved in diethyl ether (Et₂O) (60 mL) at ambient temperature, followed by the addition of dimethylformamide (DMF) (0.030 mL). After addition of oxalyl chloride (2.03 mL, 1.2 eq.) the mixture was stirred for 30 min at ambient temperature. Gas evolution is gradually ceasing during that period of time. The solvents were evaporated under reduced pressure yielding the crude product, which was sufficiently pure to be subjected to the next step.

### Example 2: Synthesis of (4,6-dichloropyrimidin-5-yl)(4-phenoxyphenyl)methanone

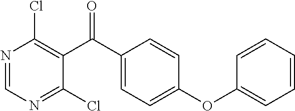 [SMILES: O=C(c1ccc(Oc2ccccc2)cc1)c1c(Cl)ncnc1Cl]

In a three-necked round-bottom flask with nitrogen-inlet, the acid chloride prepared in Example 1 was dissolved in CH₂Cl₂ (220 mL). AiCl₃ (7.25 g, 2.5 eq.) and diphenyl ether (8.97 mL, 2.6 eq.) were added, resulting in a pale yellow suspension. The reaction mixture was stirred at 50° C. overnight and subsequently poured on 400 mL ice-water. The phases were separated and the aqueous layer was extracted with CH₂Cl₂ (1×). The combined organic layers were washed with H₂O (2×), saturated NaCl-solution (2×), dried over anhydrous sodium sulfate, filtered and evaporated. The crude product was purified using column chromatography (cyclohexane/ethyl acetate 25:1), yielding the product as a colourless, crystalline solid. The product was identified by ¹H NMR giving the following peaks:

¹H NMR (500 MHz, d⁶-DMSO) δ=9.14 (s, 1H), 8.01 (d, J=8.85 Hz, 2H), 7.50 (d, J=7.95 Hz, 2H), 7.31 (t, J=7.45 Hz, 1H), 7.22 (d, J=7.65 Hz, 2H), 7.09 (d, J=8.90 Hz, 2H).

### Example 3: Synthesis of (4-amino-6-chloropyrimidin-5-yl)(4-phenoxyphenyl)methanone

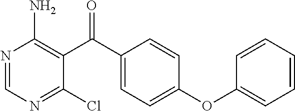 [SMILES: Nc1ncnc(Cl)c1C(=O)c1ccc(Oc2ccccc2)cc1]

In a Schlenk-type flask, the ketone prepared in Example 2 (1.0 g, 1 eq.) was dissolved in toluene (70 mL). The reaction vessel was evacuated and backfilled with N₂ three times before an ultimate evacuation and backfilling with NH₃ (balloon). The reaction mixture was vigorously stirred overnight at 60° C. Afterwards, the solvent was evaporated under reduced pressure yielding the product as an almost colourless, crystalline solid. The product was identified by ¹H NMR giving the following peaks:

¹H NMR (500 MHz, C₆D₆) δ=8.10 (s, 1H), 7.37 (d, J=8.85 Hz, 2H), 6.82 (t, J=7.93 Hz, 2H), 6.68 (t, J=7.43 Hz, 1H), 6.64-6.61 (m, 2H), 6.50 (d, J=8.85 Hz, 2H), 4.65 (br s, 2H).

### Example 4: Synthesis of 1-cyclohexyl-3-(4-phenoxyphenyl)-1H-pyrazolo[3,4-d]pyrimidin-4-amine

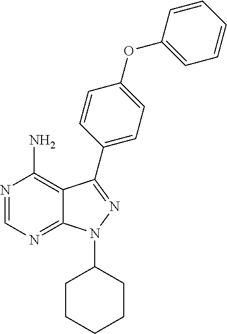 [SMILES: Nc1ncnc2c1c(-c1ccc(Oc3ccccc3)cc1)nn2C1CCCCC1]

In a screw-capped vial the ketone prepared in Example 3 (1 eq.) and cyclohexyl hydrazine hydrochloride (4.5 eq.) were suspended in 2-methyl tetrahydrofurane (THF).

After addition of triethylamine the reaction mixture was stirred overnight at 95° C. The solvent was evaporated under reduced pressure. The crude material was purified by column chromatography (toluene/ethyl acetate 1:2) yielding the product as a yellow oil which finally solidified. The product was identified by ¹H NMR and ¹³C NMR giving the following peaks:

¹H NMR (500 MHz, C₆D₆) δ=8.65 (s, 1H), 7.54 (d, J=8.60 Hz, 2H), 7.08 (t, J=7.90 Hz, 2H), 7.00-6.94 (m, 4H), 6.89 (t, J=7.33 Hz, 1H), 5.02-4.86 (m, 3H), 2.28-2.16 (m, 2H), 2.05 (d, J=11.35 Hz, 2H), 1.66 (d, J=13.15 Hz, 2H), 1.50-1.03 (m, 4H).

¹³C NMR (125 MHz, C₆D₆) δ=158.4, 158.2, 157.3, 156.1, 154.7, 143.2, 130.4, 130.2, 129.5, 124.0, 119.7, 119.4, 99.0, 56.5, 32.7, 25.8, 25.7.

### Example 5: Synthesis of 3-(4-phenoxyphenyl)-1H-pyrazolo[3,4-d]pyrimidin-4-amine

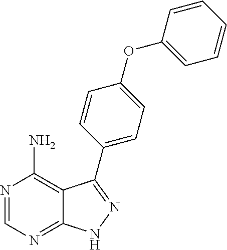 [SMILES: Nc1ncnc2[nH]nc(-c3ccc(Oc4ccccc4)cc3)c12]

In a screw-capped vial the ketone of Example 3 (100 mg, 1 equiv) and hydrazine-solution in THF (1M, 1.38 mL, 4.5 eq.) were suspended in 2-methyl THF. After addition of triethylamine the reaction mixture was stirred for 2 h at 95° C. The mixture is cooled to ambient temperature, the precipitate collected by vacuum filtration yielding the product as a crystalline solid.

1. A process for the preparation of a compound of formula (I), 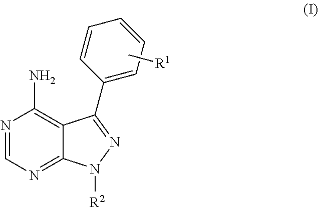
comprising reacting a compound of formula (II) 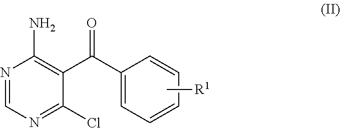
with a compound of formula (III) 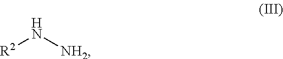
optionally in the presence of an alkaline substance, wherein
R¹ is selected from OR⁴, SR⁴, NR⁴R⁵ and halogen,
R² is selected from hydrogen, substituted or unsubstituted alkyl, substituted or unsubstituted cycloalkyl and substituted or unsubstituted heterocycloalkyl, and
R⁴ and R⁵ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl.

2. The process according to claim 1 wherein R¹ is OR⁴ and R⁴ is a substituted or non-substituted aryl.

3. The process according to claim 2 wherein R¹ is OPh.

4. The process according to claim 1, wherein R² is selected from substituted or unsubstituted cycloalkyl and substituted or unsubstituted heterocycloalkyl.

5. The process according to claim 1, wherein the compound according to formula (III) is represented by the following formula (IIIa) 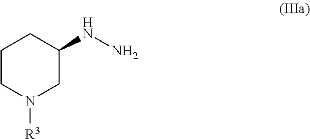
wherein R³ is selected from hydrogen, a group selected from carbamoyl, substituted or non-substituted benzyl and substituted or non-substituted silyl, and C(O)—R⁶, wherein
R⁶ is selected from hydrogen, substituted or non-substituted alkyl, substituted or non-substituted alkenyl, substituted or non-substituted heteroalkyl, substituted or non-substituted heteroalkenyl, substituted or non-substituted cycloalkyl, substituted or non-substituted heterocycloalkyl, substituted or non-substituted cycloalkenyl, substituted or non-substituted heterocycloalkenyl, substituted or non-substituted aryl, and substituted or non-substituted heteroaryl.

6. The process according to claim 1, wherein the compound according to formula (III) is represented by the following formula (IIIb)

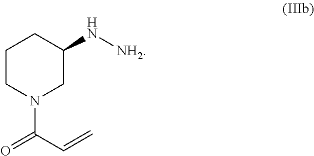

7. The process according to claim 1, wherein the compound according to formula (II), 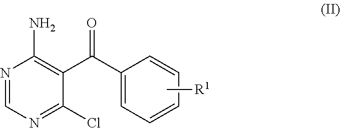
is obtained by monoamination of a compound of formula (IV)

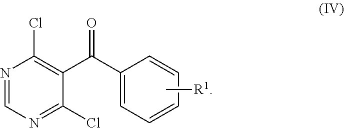

8. The process according to claim 7, wherein monoamination is carried out in the presence of ammonia and with a temperature in a range of 20 to 100° C.

9. The process according to claim 7, wherein the compound according to formula (IV) is obtained by reacting a compound according formula (V) 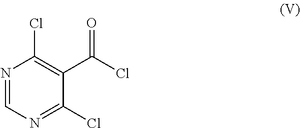 [SMILES: O=C(Cl)c1c(Cl)ncnc1Cl]
with a compound according to formula (VI) 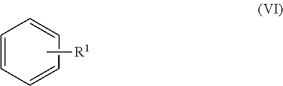
in the presence of a Lewis acid.

10. The process according to claim 9, wherein the compound according to formula (V) is obtained by reacting a compound according formula (VII) 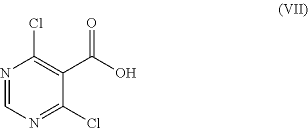 [SMILES: O=C(O)c1c(Cl)ncnc1Cl]
with a chlorinating agent.

11. The process according to claim 10, wherein the chlorinating agent is selected from one or more of (COCl)₂/DMF, SOCl₂, PCl₅, PCl₃, POCl₃/DMF, 1-Chloro-N,N,2-trimethyl-1-propenylamine.

12. The process according to claim 1, wherein R¹ is OPh, and compound (III) is represented by the compound of formula (IIIa), to obtain a compound represented by formula (Ia)

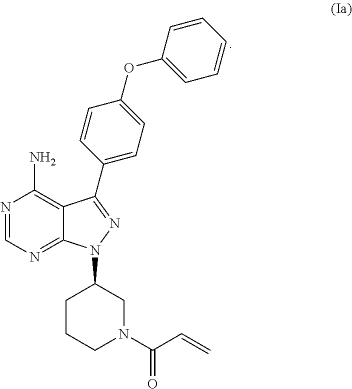 [SMILES: C=CC(=O)N1CCC[C@@H](n2nc(-c3ccc(Oc4ccccc4)cc3)c3c(N)ncnc32)C1]

13. A compound represented by the formula (IIa)

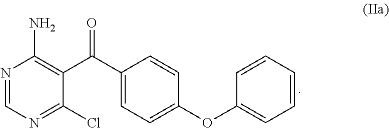 [SMILES: Nc1ncnc(Cl)c1C(=O)c1ccc(Oc2ccccc2)cc1]

14. (canceled)
